(19) United States
(12) Patent Application Publication
(10) Pub. No.: US 2008/0026073 A1
(43) Pub. Date: Jan. 31, 2008

(54) METHOD AND COMPOSITION FOR LITHIUM ADDITIVE IN TREATMENT OF GLUTAMATE TOXICITY

(76) Inventors: William R. Peitzke, Montecito, CA (US); George Ayoub, Santa Barbara, CA (US)

Correspondence Address: FELIX L. FISCHER, ATTORNEY AT LAW, 1607 MISSION DRIVE, SUITE 204, SOLVANG, CA, 93463, US

(21) Appl. No.: 11/771,355

(22) Filed: Jun. 29, 2007

Related U.S. Application Data
(60) Provisional application No. 60/806,256, filed on Jun. 29, 2006.
(60) Provisional application No. 60/806,611, filed on Jul. 5, 2006.

(51) Int. Cl. A61K 33/00; A61P 25/00

(52) U.S. Cl. 424/610

(57) ABSTRACT

Treatment of glutamate toxicity is accomplished using Lithium or a composition of Lithium and a botanical extract. The composition is administered using conventional techniques to increase the survival of ganglion cells exposed to glutamate toxicity resulting from ischemic events. In exemplary embodiments, the botanical extract is St. Johns Wort, Tian Qi or Dong Gui.

### REFERENCE TO RELATED APPLICATIONS

This patent application claims benefit of priority of provisional applications Ser. No. 60/806,256 filed on Jun. 29, 2006 and Ser. No. 60/,806,611 filed on Jul. 5, 2006 both having the same title as the present application and a common assigned with the present application. The disclosure of the provisional applications is incorporated herein by reference.

### BACKGROUND OF THE INVENTION

1. Field of the Invention

This invention relates generally to the field of nutraceuticals for the treatment of neurological disorders and more particularly to the use of Lithium and a combination of botanical extracts or analogs thereof with specific metals or metal compounds such as Lithium for treatment of glutamate toxicity.

2. Description of the Related Art

Glaucoma is an exemplary neurological disorder that is only recently becoming understood. The current treatment for Glaucoma consists of medicines and surgical procedures intended to slow the onset of the disease by decreasing intra-ocular pressure (IOP). While this slows the process which ultimately leads to retinal damage, it does not block the final mechanism of cell death.

The mechanism of neural cell death in retinal tissue can be attributed to a series of ischemic events in which the circulation of blood to the retina is interrupted or impaired due to several possible conditions. A high IOP generally results in a lower perfusion pressure into the eye. This condition can be exacerbated by other medical conditions. The net effect is a series of small stroke-like events that in time lead to a toxic buildup of the neurotransmitter glutamate.

The process, referred to as “glutamate toxicity,” occurs when the normal mechanisms for removing the neurotransmitter glutamate have been impaired or excess glutamate otherwise becomes present. Indeed, neural degenerative diseases often involve glutamate toxicity. While glutamate is a naturally occurring neural transmitter, when its concentration is uncontrolled it becomes highly toxic. In glaucoma patients, glutamate increases in concentration, eventually leading to neural damage of the optic nerve. This buildup of excess glutamate has been determined to trigger cell death; nerve cells and glial cells deprived of blood flow fail to remove the released glutamate, as a result it remains in the tissue triggering a cascading cell death along the nerve.

The use of botanical derivatives in the treatment of glaucoma has been previously reported, particularly the use of cannabinoids as disclosed in U.S. patent publication 20020077322 dated Feb. 20, 2002. In general, it is understood that by increasing the activity of a cannabinoid agonist that binds specifically to an endogenous cannabinoid receptor, cells of the nervous system, such as ganglion cells, may be protected against glutamate-induced neurotoxicity. The efficacy of such treatment is not as high as desired and the use of cannabinoid-derived nutraceuticals is burdened with legal as well as technical challenges.

It is therefore desirable to provide a treatment that is based on the chemical sequence of events leading to cell death in circulation-impaired neural tissue and to interrupt it.

### SUMMARY OF THE INVENTION

The present invention incorporates a composition for treatment of glutamate toxicity employing Lithium and combinations of a botanical extract and Lithium. Administration using conventional techniques is employed to increase the survival of nerve cells exposed to glutamate toxicity resulting from ischemic events. In exemplary embodiments, the botanical extract is St. Johns Wort, Tian Qi or Dong Gui.

### BRIEF DESCRIPTION OF THE DRAWINGS

These and other features and advantages of the present invention will be better understood by reference to the following detailed description when considered in connection with the accompanying drawings wherein:

FIG. 1 is graphical depiction of the increase in surviving retinal ganglion cells in test conditions measured with the use of Lithium and varying combinations of Lithium and St. Johns Wort;

FIG. 2 is a graphical depiction of the increase in retinal ganglion cells measured with the use of Lithium and varying combinations of Lithium and botanical extracts of St. John's Wort, Tian Qi and Dong Gui; and,

FIG. 3 is a graphical depiction of the survival of retinal ganglion cells in test conditions with varying concentrations of Lithium alone.

### DETAILED DESCRIPTION OF THE INVENTION

The compositions and methods employed in the present invention disclosed herein incorporate formulations for treatment using Lithium in the form of various lithium compounds such as lithium orotate and botanical extracts combined with Lithium to increase the survival of retinal ganglion cells exposed to ischemic events resulting in glutamate toxicity as occurs in glaucoma. The compositions can be administered using a number of different routes including orally, topically, transdermally, transclerally, transepithelially, intraocularly, intravitreally, enteral administration, administration by intraperitoneal injection or administration by intravenous injection directly into the bloodstream. The compositions to be used can also be administered via transmucosal application, such as by a nasal spray, inhaler, or by sublingual application. Effective amounts of the combination nutraceutical can also be administered through injection into the cerebrospinal fluid or infusion directly into the brain, if desired. Composition percentages will vary based on the administration technique. The administration route for the exemplary data provided herein is oral ingestion based on current packaging approaches. However, for glaucoma treatment as envisioned, the administration is anticipated to be via eye drops or a nasal spray. Drops are the conventional route for glaucoma medications currently.

Exemplary formulations for administration of Lithium in human equivalent dosage of Lithium provided by 5.8 mg of lithium orotate per day and botanical compositions according to the invention are prepared with St. Johns Wort and Lithium in human equivalent dosages of St. John's Wort at 2-4 grams per day of crude herb, which is equivalent to standardized extract containing 900 mg of hypericin. In exemplary testing, the compositions were administered to test subject mice by oral ingestion and toxic retinal conditions such as those found in glaucoma were induced. The count of the number of surviving retinal ganglion cells was shown to increase for the tested compositions by the percentage of cells that survived due to treatment i.e. that did not survive when no treatment was present. The rod is the error range in the sample.

### EXAMPLE 1

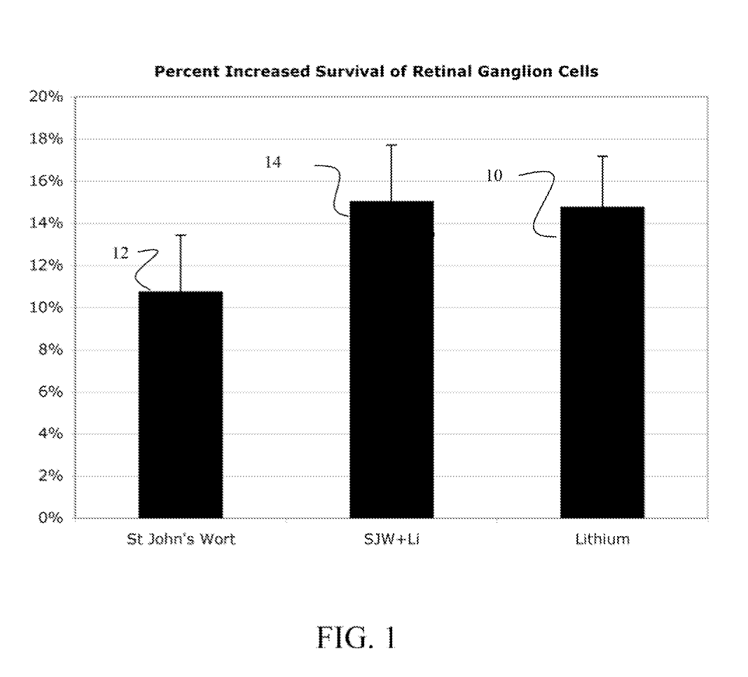

As shown in FIG. 1, Lithium and various combinations of Lithium and St. John's Wort have varying efficacy. Lithium alone has an efficacy yielding an increase in the surviving ganglion cells of about 15%, bar 10, while St. Johns Wort alone yields about 11%, bar 12. The combined composition using Lithium and St. Johns Wort in the previously described 2-4 gram dosage with Lithium in the 5.8 mg dosage demonstrates an increase in surviving ganglion cells also of about 15%, bar 14. This first study compares Lithium and an exemplary composition incorporating St. Johns Wort as the botanical.

### EXAMPLE 2

Alternative botanical ingredients to St. Johns Wort, Tian Qi (aka Tien Chi or Tien Qi) and Dong Gui (also known as Dong Gui or Tang Kuai) are employed for other embodiments. In an exemplary use at dosages comparable the prior disclosed human dosage for St. Johns Wort the following protocol for mouse testing has been adopted. Tian Qi is employed in the formulations as crude powder at 2-3 grams per day to provide the maximum efficacy.

Mice are fed compounds of a botanical individually and in combination with lithium via gavage for a duration of eight days. After the first six of those days, each mouse is given an injection of NMDA into one vitreal chamber, and an injection of saline vehicle into the other vitreal chamber. A one-half microliter volume of a solution containing 150 mM NMDA (or physiological saline) is injected. Two days later the mice are euthanized by an intraperitoneal injection of sodium pentobarbital followed by perfusion fixation. Retinas are dissected, stained with cresyl violet, and the number of retinal ganglion cells surviving counted.

Lithium is fed to the mice at a dose of 63 μg/day (Li orotate) calculated to be the equivalent of the human dosage described above based on body mass and metabolism, being made up in solution for introduction. In certain embodiments, Lithium alone is employed while in alternative embodiments, the three medicinal herbs are fed to the animals, with these herbs being provided by a medicinal herbalist that uses them in the treatment of humans. The compounds are created with St. John's Wort, Tian Qi, and Dang Gui and Lithium in conjunction with these herbs. For the three herbs, the preparation calls for them to be crushed and then suspended in water, at a concentration of 6% w/v. Each mouse is then fed 0.5 ml of this suspension each day for the duration of the course. A concentration of 32 mg of herb per mouse per day is provided. These dosages were determined by using the human dosages used for each element of the composition, and then scaling this based on the difference in mass between a human and mouse and the difference in metabolic rates of the two.

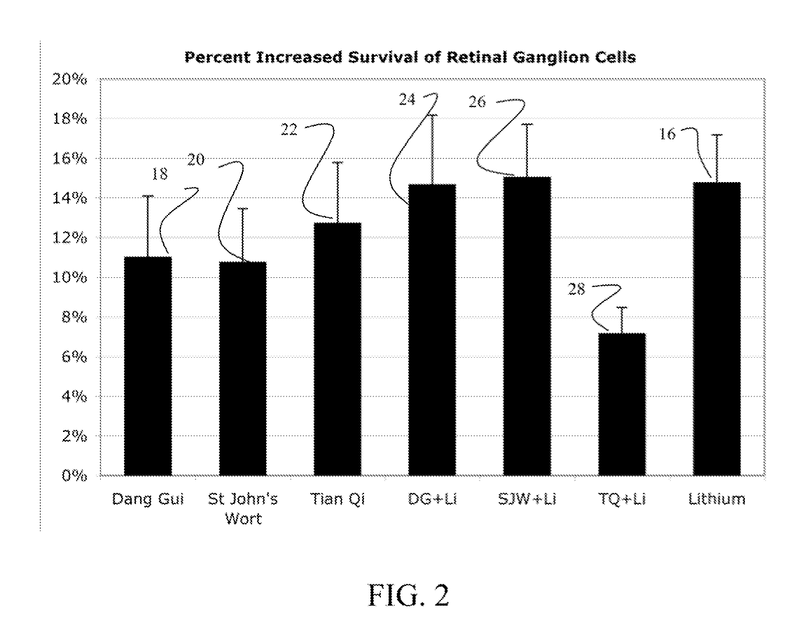

The test results summarized in FIG. 2 show that Lithium alone again provides a survival rate of retinal ganglion cells of about 15%, bar 16. The botanical ingredients used individually provide survival rates of approximately 11% for Dang Gui, bar 18, 11% for St. John's Wort, bar 20 and 13% for Tian Qi, bar 22. The formulations providing a combined compound demonstrate an enhanced effect with Dang Gui and Lithium at approximately 15%, bar 24, and St. John's Wort and Lithium at approximately 15%, bar 26. A slight decline in efficacy for the combination of Tan Qi and Lithium of approximately 7% was noted bar 28.

### EXAMPLE 3

As demonstrated in the prior examples, Lithium alone provides an efficacious treatment for increasing the survival of retinal ganglion cells exposed to ischemic events resulting in glutamate toxicity as occurs in glaucoma.

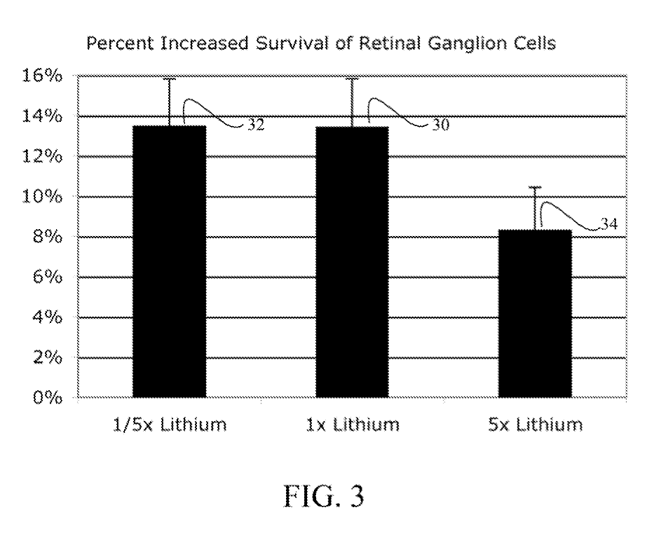

As a comparative example run as a separate experiment with different mouse subject pools from the prior examples, three dosages of Lithium as a neuroprotectant in the mouse model for glaucoma described above are employed. NMDA was used to elicit glutamate toxicity, and the animals are fed the Lithium formulation. The numbers of surviving retinal ganglion cells are counted and a percent is calculated based on the number of cells that survive relative to no treatment. As shown in FIG. 3, dosage modification are compared from the baseline Lithium dosage of 5.8 mg, bar 30, previously employed in the first two examples. At ⅕^{th} the dosage, bar 32, corresponding to a human Lithium dosage equivalent of 1.16 mg lithium oronate, provides comparable survival efficacy of approximately 13% while an increase in the dosage to 5 times, bar 26, a human Lithium dosage equivalent of 29 mg lithium orotate, creates a slight reduction in efficacy to approximately 8%.

A range of effective compositions for Lithium is therefore demonstrated to be at least 1.16 mg to 29 mg of lithium orotate as a human dosage equivalent.

The methods of the present invention can be effected using Lithium or the botanical/Lithium compositions administered to a mammalian subject either alone or in combination as a pharmaceutical formulation. Further, the Lithium or botanical/Lithium compositions can be combined with pharmaceutically acceptable excipients and carrier materials such as inert solid diluents, aqueous solutions or non-toxic organic solvents. If desired, these pharmaceutical formulations can also contain preservatives and stabilizing agents and the like, as well as minor amounts of auxiliary substances such as wetting or emulsifying agents, as well as pH buffering agents and the like which enhance the effectiveness of the active ingredient. The pharmaceutically acceptable carrier can be chosen from those generally known in the art including, but non limited to, human serum albumin, ion exchangers, dextrose, alumina, lecithin, buffer substances such as phosphate, glycine, sorbic acid, potassium sorbate, propylene glycol, polyethylene glycol, and salts or electrolytes such as protamine sulfate, sodium chloride, or potassium chloride. Other carriers can be used.

Liquid compositions can also contain liquid phases either in addition to or to the exclusion of water. Examples of such additional liquid phases are glycerin, vegetable oils such as cottonseed oil, organic esters such as ethyl oleate, and water-oil emulsions.

The compositions can be made into aerosol formulations (i.e., they can be “nebulized”) to be administered via inhalation. Aerosol formulation can be placed into pressurized acceptable propellants, such as dichloromethane, propane, or nitrogen. Other suitable propellants are known in the art

Formulations suitable for enteral administration include aqueous and non-aqueous, isotonic sterile solutions. These can contain antioxidants, buffers, preservatives, bacteriostatic agents, and solutes that render the formulation isotonic with the blood or fluid of the particular recipient as required. Alternatively, these formulations can be aqueous or non-aqueous sterile suspensions that can include suspending agents, thickening agents, solubilizers, stabilizers, and preservatives. Preparation of solutions for enteral administration is well known in the art and need not be described further here.

Formulations suitable for parental administration, such as, for example, by intravenous, intraocular, intravitreal, intramuscular, intradermal, and subcutaneous routes, include aqueous and non-aqueous, isotonic sterile injection solutions. These can contain antioxidants, buffers, preservatives, bacteriostatic agents, and solutes that render the formulation isotonic with the blood or fluid of the particular recipient as required. Alternatively, these formulations can be aqueous or non-aqueous sterile suspensions that can include suspending agents, thickening agents, solubilizers, stabilizers, and preservatives. Compositions suitable for use in methods according to the present invention could be administered, for example, by intravenous infusion, orally, topically, transdermally, intraocularly, intravitreally, transepithelially, transclerally, intraperitoneally, intravesically, or intrathecally. Formulations of compounds suitable for use in methods according to the present invention can be presented in unit-dose or multi-dose sealed containers, in physical form such as ampoules or vials.

Having now described the invention in detail as required by the patent statutes, those skilled in the art will recognize modifications and substitutions to the specific embodiments disclosed herein. Such modifications are within the scope and intent of the present invention as defined in the following summary statements.

What is claimed is:

1. A method for treatment of glutamate toxicity comprising the steps of:
preparing a formulation containing Lithium in a human dosage equivalent to between about 1.16 mg lithium orotate and 29 mg lithium orotate;
administering the formulation.

2. The method as defined in claim 1 wherein the step of administering comprises oral ingestion of the formulation.

3. The method as defined in claim 1 wherein the step of administering comprises topical application to the eye.

4. The method as defined in claim 1 wherein the step of administering comprises transmucosal application.

5. The method as defined in claim 1 wherein the step of administering is selected from the administration techniques comprising transdermal administration, transcleral administration transepithelial administration, intraocular administration, intravitreal administration, enteral administration, administration by intraperitoneal injection or administration by intravenous injection directly into the bloodstream and the step of preparing includes adjustment of formulation dosage for greatest efficacy in the selected administration technique.

6. A composition for treatment of glutamate toxicity comprising
Lithium in a human dosage equivalent to between about 1.16 mg lithium orotate and 29 mg lithium orotate, and,
a botanical.

7. A composition as defined in claim 6 wherein the Lithium is mixed with the botanical in exact form at about 0.6 percent weight.

8. A composition as defined in claim 6 wherein the botanical is St. Johns Wort.

9. A composition as defined in claim 6 wherein the botanical is Tian Qi.

10. A composition as defined in 2 wherein the botanical is Dong Gui.

11. A method for treatment of glutamate toxicity comprising the steps of:
mixing a composition of a Lithium and a botanical;
administering the mixed composition.

12. A method as defined in claim 11 wherein the step of administering comprises oral ingestion of the composition.

13. A method as defined in claim 11 wherein the step of mixing comprises combining about 3 grams of raw botanical and about 5.8 miligrams of Lithium.

14. A method as defined in 11 wherein the botanical extract is St. Johns Wort.

16. A method as defined in claim 11 wherein the botanical extract is Tian Qi.

17. A method as defined in claim 11 wherein the botanical is in extract form and the step of mixing comprises containing about 900 milligrams of extract with about 5.8 grams of Lithium.

18. A method as defined in claim 17 wherein the Lithium is in the form of Lithium orotate.

19. The method as defined in claim 13 wherein the step of administering is selected from the administration techniques comprising topical administration, transdermal administration, transcleral administration, transepithelial administration, intraocular administration, intravitreal administration, enteral administration, administration by intraperitoneal injection or administration by intravenous injection directly into the bloodstream and the step of mixing includes adjustment of formulation dosage in the composition for greatest efficacy, in the selected administration technique.

20. A pharmaceutical formulation for treatment of glutamate toxicity comprising Lithium in an efficacious dosage.
